(19) United States
(12) Patent Application Publication
(10) Pub. No.: US 2026/0337493 A1
(43) Pub. Date: Oct. 29, 2026

(54) METHODS FOR REACTING HYDROCARBONS UTILIZING STRIPPERS

(71) Applicant: Dow Global Technologies LLC, Midland, MI (US)

(72) Inventors: Matthew T. Pretz, Lake Jackson, TX (US); Davy Nieskens, Terneuzen (NL)

(73) Assignee: Dow Global Technologies LLC, Midland, MI (US)

(21) Appl. No.: 19/111,469

(22) PCT Filed: Sep. 13, 2023
(86) PCT No.: PCT/US2023/074024

§ 371 Date: Mar. 13, 2025

Related U.S. Application Data
(60) Provisional application No. 63/406,445, filed on Sep. 14, 2022.

(51) Int. Cl. C10G 3/00

(52) U.S. Cl. CPC C10G 3/49; C10G 3/62; C10G 2300/708

(57) ABSTRACT

According to one or more embodiments described herein, olefinic compounds may be formed by a process including contacting a feed stream including one or more hydrocarbons with a particulate solid in a reactor. In the reactor, the one or more hydrocarbons may be reacted to form one or more olefinic compounds and coke may form on the particulate solid. The particulate solid may be passed from the reactor to a strip zone. In the strip zone, the particulate solid may be contacted with a strip gas and oxygen. The strip gas may include nitrogen, steam, or combinations thereof. In the strip zone, a majority of the coke may be reacted with oxygen to form carbon dioxide. The majority of the carbon dioxide produced in the strip zone may be passed out of the strip zone and combined with the one or more olefinic compounds.

### CROSS-REFERENCE TO RELATED APPLICATIONS

This application claims the benefit of U.S. Provisional Application Ser. No. 63/406,445 filed Sep. 14, 2022, the entire disclosure of which is hereby incorporated herein by reference.

### TECHNICAL FIELD

Embodiments described herein generally relate to chemical processing and, more specifically, to processes and systems utilized for the formation of olefinic compounds.

### BACKGROUND

Olefinic compounds may be utilized as base materials to produce many types of goods and materials. For example, ethylene may be utilized to manufacture polyethylene, ethylene chloride, or ethylene oxides. Such products may be utilized in product packaging, construction, textiles, etc. Thus, there is an industry demand for olefinic compounds, such as ethylene, propylene, butene, and styrene.

### SUMMARY

Olefinic compounds may be formed from various hydrocarbon feeds. For example, olefinic materials may be formed by the dehydrogenation of alkanes (catalytic or thermal). In some of these processes, particulate solids are utilized, which may generally cycle between a reactor and a regeneration unit. In such processes, coke may form on the particulate solid. In many conventional processes, the coke is combusted in the regeneration unit, forming CO₂ that is eventually expelled to the environment. This is not ideal, since CO₂ is a known greenhouse contributing gas. As disclosed herein, coke in such processes may be reacted in a strip zone with oxygen to form CO₂, which is combined with the olefinic products. In one or more embodiments, the CO₂ may be subsequently separated from the olefinic products and not released into the environment.

According to one or more embodiments described herein, olefinic compounds may be formed by a process comprising contacting a feed stream comprising one or more hydrocarbons with a particulate solid in a reactor. In the reactor the one or more hydrocarbons may be reacted to form one or more olefinic compounds and coke may form on the particulate solid. The process may further comprise passing the particulate solid from the reactor to a strip zone. In the strip zone the particulate solid may be contacted with a strip gas and oxygen. The strip gas may comprise nitrogen, steam, or combinations thereof. In the strip zone a majority of the coke may be reacted with oxygen to form carbon dioxide. The majority of the carbon dioxide produced in the strip zone may be passed out of the strip zone and combined with the one or more olefinic compounds. The process may further comprise passing the particulate solid from the strip zone to a regeneration unit, wherein the particulate solid may be heated by combustion of a fuel in the regeneration unit. The process may further comprise passing at least a portion of the particulate solid from the regeneration unit to the reactor.

It is to be understood that both the preceding general description and the following detailed description describe various embodiments and are intended to provide an overview or framework for understanding the nature and character of the claimed subject matter. Additional features and advantages of the embodiments will be set forth in the detailed description and, in part, will be readily apparent to persons of ordinary skill in the art from that description, which includes the accompanying drawings and claims, or recognized by practicing the described embodiments. The drawings are included to provide a further understanding of the embodiments and, together with the detailed description, serves to explain the principles and operations of the claimed subject matter. However, the embodiments depicted in the drawings are illustrative and exemplary in nature, and not intended to limit the claimed subject matter.

### BRIEF DESCRIPTION OF THE DRAWINGS

Reference will now be made in greater detail to various embodiments, some of which are illustrated in the accompanying drawings, wherein:

FIG. 1 schematically depicts a reactor system, according to one or more embodiments of the present disclosure; and

FIG. 2 schematically depicts another reactor system, according to additional embodiments of the present disclosure.

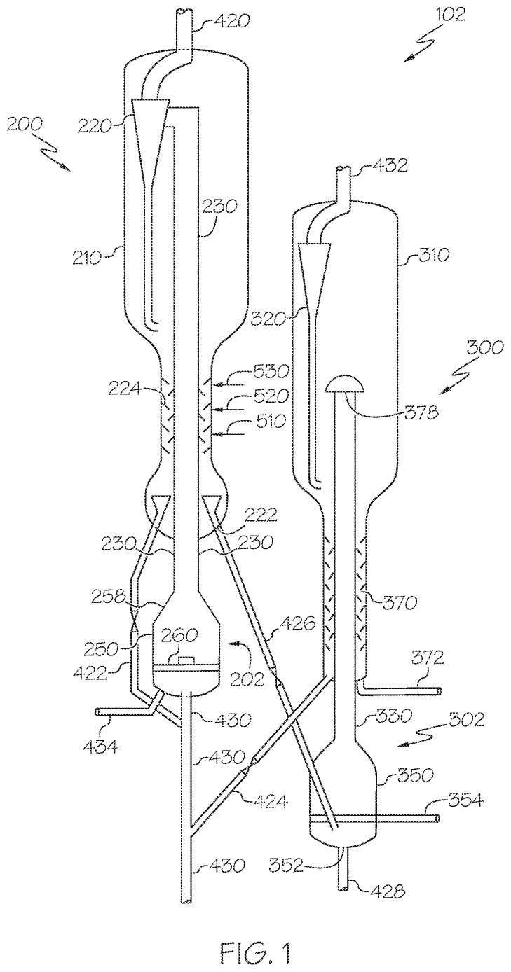

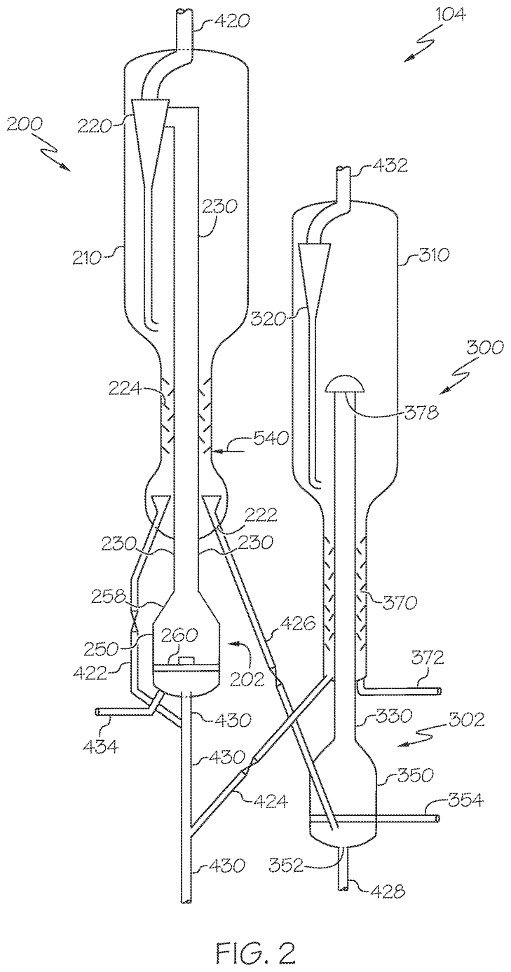

When describing the simplified schematic illustration of FIG. 1 and FIG. 2, the numerous valves, temperature sensors, electronic controllers, and the like, which may be used and are well known to a person of ordinary skill in the art, are not included. Further, accompanying components that are often included in such reactor systems, such as air supplies, heat exchangers, surge tanks, and the like are also not included. However, it should be understood that these components are within the scope of the present disclosure.

### DETAILED DESCRIPTION

Specific embodiments of the present application will now be described. The disclosure may, however, be embodied in different forms and should not be construed as limited to the embodiments set forth in this disclosure. Rather, these embodiments are provided so that this disclosure will be thorough and complete, and will fully convey the scope of the subject matter to those skilled in the art.

Embodiments presently disclosed are described in detail herein in the context of the reactor systems of FIG. 1 and FIG. 2 operating as a fluidized dehydrogenation reactor system to produce light olefins, such as propylene. However, it should be understood that the principles disclosed and taught herein may be applicable to other systems which utilize different system components oriented in different ways, or different reaction schemes utilizing various catalyst compositions. For example, the concepts described may be equally applied to other systems with alternate reactor units and regeneration units, such as those that operate under non-fluidized conditions or include downers rather than risers. It should be further understood that not all portions of the reactor systems of FIG. 1 and FIG. 2 should be construed as essential to the claimed subject matter.

Now referring to FIG. 1, an example reactor system 102 that may be suitable for use with the methods and/or apparatuses described herein is schematically depicted. The reactor system 102 generally comprises multiple system components, such as a reactor portion 200 and a regeneration unit 300. As described herein, “system components” refer to portions of the reactor system 102, such as reactors, separators, transfer lines, combinations thereof, and the like. As used herein in the context of FIG. 1, the reactor portion 200 generally refers to the portion of a reactor system 102 in which the major process reaction takes place (e.g., dehydrogenation) to form the product stream. A feed stream enters the reactor portion 200, is converted to a product stream (containing product and unreacted feed), and exits the reactor portion 200. The reactor portion 200 comprises a reactor 202 which may include an upstream reactor section 250 and a downstream reactor section 230. According to one or more embodiments, as depicted in FIG. 1, the reactor portion 200 may additionally include a particulate solid separation section 210, which serves to separate the particulate solid from the chemical products formed in the reactor 202. The particulate solid may pass through a strip zone 224 before being passed to the regeneration unit 300. In the strip zone 224 an oxygen-containing gas may enter into the strip zone 224 through a first gas inlet 520. A strip gas may enter into the strip zone 224 through a second gas inlet 510. The particulate solid may be exposed to the oxygen-containing gas in the strip zone 224 before being passed to the regeneration unit 300. Also, as used herein, the regeneration unit 300 generally refers to the portion of the reactor system 102 where the particulate solid is in some way processed, such as by combustion, to, e.g., improve catalytic activity and/or heat the particulate solid. The regeneration unit 300 may comprise a combustor 350 and a riser 330, and may additionally comprise a particulate solid separation section 310. In one or more embodiments, the particulate solid separation section 210 may be in fluid communication with the combustor 350 (e.g., via standpipe 426) and the particulate solid separation section 310 may be in fluid communication with the upstream reactor section 250 (e.g., via standpipe 424 and transport riser 430).

Generally as is described herein, in embodiments illustrated in FIG. 1 and FIG. 2, the particulate solid is cycled between the reactor portion 200 and the regeneration unit 300. It should be understood that when particulate solids are referred to herein, they may refer to solid materials that are catalytically active for a desired reaction, or may equally refer to other particulate solids referenced with respect to the systems of FIG. 1 and FIG. 2 which do not necessarily have catalytic activity but affect the reaction, such as oxygen-carrier materials. The terms “catalytic activity” and “catalyst activity” refer to the degree to which the particulate solid is able to catalyze the reactions conducted in the reactor system 102. The particulate solid that exits the reactor portion 200 may be deactivated particulate solid. As used herein, “deactivated” may refer to a particulate solid which has reduced catalytic activity or is cooler as compared to particulate solid entering the reactor portion 200. However, deactivated particulate solid may maintain some catalytic activity. Reduced catalytic activity may result from contamination with a substance such as coke. Reactivation (sometimes called “regeneration” herein) may remove the contaminant such as coke, raise the temperature of the particulate solid, or both. In embodiments, a majority of the coke formed on the particulate solid may be reacted with oxygen in the strip zone 224 before the particulate solid is passed to the regeneration unit 300. In embodiments, deactivated particulate solid may be reactivated by particulate solid reactivation in the regeneration unit 300. The deactivated particulate solid may be reactivated by, but not limited to, removing coke by combustion, recovering catalyst acidity, oxidizing the particulate solid, other reactivation process, or combinations thereof. In some embodiments, the particulate solid may be heated during reactivation by combustion of a fuel, such as hydrogen, methane, ethane, propane, natural gas, or combinations thereof. The reactivated particulate solid from the regeneration unit 300 may then be passed back to the reactor portion 200.

The feed stream may enter feed inlet 434 into the reactor 202, and the product stream may exit the reactor system 102 via pipe 420. According to one or more embodiments, the reactor system 102 may be operated by feeding a chemical feed (e.g., in a feed stream) and a fluidized particulate solid into the upstream reactor section 250. The chemical feed contacts the particulate solid in the upstream reactor section 250, and each flow upwardly into and through the downstream reactor section 230 to produce a chemical product.

Now referring to FIG. 1 in detail, the reactor portion 200 may comprise an upstream reactor section 250, a transition section 258, and a downstream reactor section 230, such as a riser. The transition section 258 may connect the upstream reactor section 250 with the downstream reactor section 230. As depicted in FIG. 1, the upstream reactor section 250 may be positioned below the downstream reactor section 230. Such a configuration may be referred to as an upflow configuration in the reactor 202. The upstream reactor section 250 may include a vessel, drum, barrel, vat, or other container suitable for a given chemical reaction. As depicted in FIG. 1, the upstream reactor section 250 may be connected to the downstream reactor section 230 via the transition section 258. The upstream reactor section 250 may generally comprise a greater cross-sectional area than the downstream reactor section 230. The transition section 258 may be tapered from the size of the cross-section of the upstream reactor section 250 to the size of the cross-section of the downstream reactor section 230 such that the transition section 258 projects inwardly from the upstream reactor section 250 to the downstream reactor section 230. For example, the transition section 258 may be a frustum.

The upstream reactor section 250 may be connected to a transport riser 430, which, in operation may provide reactivated particulate solid in a feed stream to the reactor portion 200. The reactivated catalyst and/or reactant chemicals may be mixed with a distributor 260 housed in the upstream reactor section 250. The particulate solid entering the upstream reactor section 250 via transport riser 430 may be passed through standpipe 424 to a transport riser 430, thus arriving from the regeneration unit 300. In some embodiments, particulate solid may come directly from the particulate solid separation section 210 via standpipe 422 and into a transport riser 430, where it enters the upstream reactor section 250, where in such embodiments some of the particulate solid is not passed through the regeneration unit 300. The particulate solid can also be fed via standpipe 422 directly to the upstream reactor section 250 (not depicted in FIG. 1). This particulate solid may be somewhat deactivated, but may still, in some embodiments, be suitable for reaction in the upstream reactor section 250, particularly when used in combination with reactivated particulate solid.

Still referring to FIG. 1, in one or more embodiments, based on the shape, size, and other processing conditions (such as temperature and pressure) in the upstream reactor section 250 and the downstream reactor section 230, the upstream reactor section 250 may operate as a fluidized bed, such as in a fast fluidized, turbulent, or bubbling bed upflow reactor, while the downstream reactor section 230 may operate in more of a plug flow manner, such as in a riser reactor. For example, the reactor 202 of FIG. 1 may comprise an upstream reactor section 250 operating as a fast fluidized, turbulent, or bubbling bed reactor and a downstream reactor section 230 operating as a dilute phase riser reactor, with the result that the average particulate solid and gas flow moves concurrently upward. As the term is used herein, “average flow” refers to the net flow, i.e., the total upward flow minus the retrograde or reverse flow, as is typical of the behavior of fluidized particles in general. As described herein, a “fast fluidized” reactor may refer to a reactor utilizing a fluidization regime wherein the superficial velocity of the gas phase is greater than the choking velocity and may be semi-dense in operation. As described herein, a “turbulent” reactor may refer to a fluidization regime where the superficial velocity of less than the choking velocity and is more dense than the fast fluidized regime. As described herein, a “bubbling bed” reactor may refer to a fluidization regime wherein well defined bubbles in a highly dense bed are present in two distinct phases. The “choking velocity” refers to the minimum velocity required to maintain solids in the dilute-phase mode in a vertical conveying line. As described herein, a “dilute phase riser” may refer to a riser reactor operating at transport velocity, where the gas and particulate solid have about the same velocity in a dilute phase.

According to embodiments, the chemical product and the particulate solid may be passed out of the downstream reactor section 230 to a separation device 220 in the particulate solid separation section 210, where the particulate solid is separated from the chemical product, which is transported out of the particulate solid separation section 210. According to one or more embodiments, following separation from vapors in the separation device 220, the particulate solid may generally move through the strip zone 224 to the particulate solid outlet port 222 where the particulate solid is transferred out of the reactor portion 200 via standpipe 426 and into the regeneration unit 300.

According to one or more embodiments, the strip zone 224 may comprise a first gas inlet 520 and a second gas inlet 510. The strip zone 224 is an area where stripping may occur. Stripping is a process by which entrained and adsorbed products and reactants from reactor may be removed from the particulate solid through the use of a strip gas. For example, during of the dehydrogenation of ethane stripping may remove ethane, ethylene, and other hydrocarbon by products. An oxygen-containing gas may enter the strip zone 224 through the first gas inlet 520. A strip gas may enter the strip zone 224 through the second gas inlet 510. In some embodiments, the strip gas may comprise nitrogen, steam or combinations thereof.

The particulate solid may move in a generally downwards direction through the strip zone 224. As used in the present disclosure the term “generally downward direction” means that the average velocity of the particulate solid is in the downward direction, where the downward direction is with the pull of gravity. As it is an average, the velocity of individual particles of the particulate solid may have a distribution and may not be equal to the average, but taken as a whole the velocity of the particulate solid will average out to be generally downward. According to embodiments, gases within the strip zone 224 may move in a generally upwards direction through the strip zone 224. As used in the present disclosure the term “generally upward direction” means that the average velocity of gases within the strip zone 224 is in the upward direction, where the upward direction is against the pull of gravity. As it is an average, the velocity of the gas molecules within the strip zone 224 may have a distribution and may not be equal to the average, but taken as a whole the velocity of the gases will average out to be generally upward. According to embodiments, the particulate solid and the gases may move in a countercurrent flow pattern through the strip zone 224.

In one or more embodiments the first gas inlet 520 may be above the second gas inlet 510 as shown in FIG. 1. In some embodiments, the first gas inlet 520 may be below the second gas inlet 510. In some embodiments, the first gas inlet 520 and the second gas inlet 510 may be at the same height within the strip zone 224.

As described herein, in one or more embodiments an oxygen-containing gas may enter the strip zone 224 through the first gas inlet 520. In some embodiments, the oxygen-containing gas may be air, enriched air, air mixed with steam, or flue gas. Enriched air is air with added oxygen. In some embodiments, the oxygen-containing gas may include at least 28 mol. % oxygen. In other embodiments, the oxygen-containing gas may include from about 2 mol. % to about 28 mol. % oxygen, from about 2 mol. % to about 25 mol. %, from about 2 mol. % to about 20 mol. %, from about 2 mol. % to about 15 mol. %, from about 2 mol. % to about 10 mol. %, from about 2 mol. % to about 5 mol. %, from about 5 mol. % to about 28 mol. %, from about 5 mol. % to about 25 mol. %, from about 5 mol. % to about 20 mol. %, from about 5 mol. % to about 15 mol. %, from about 5 mol. % to about 10 mol. %, from about 10 mol. % to about 28 mol. %, from about 10 mol. % to about 25 mol. %, from about 10 mol. % to about 20 mol. %, from about 10 mol. % to about 15 mol. %, from about 15 mol. % to about 28 mol. %, from about 15 mol. % to about 25 mol. %, from about 15 mol. % to about 20 mol. %, from about 20 mol. % to about 28 mol. %, from about 20 mol. % to about 25 mol. %, or from about 25 mol. % to about 28 mol. %. In some embodiments, at least a portion of the coke that forms on the particulate solid may react with oxygen in the strip zone 224 to produce carbon dioxide. In some embodiments, at least 95 wt. % of the coke may be reacted in the strip zone 224. In other embodiments from about 75 wt. % to about 99 wt. % of the coke may be reacted in the strip zone 224, such as from about 75 wt. % to about 95 wt. %, from about 75 wt. % to about 90 wt. %, from about 75 wt. % to about 85 wt. %, from about 75 wt. % to about 80 wt. %, from about 80 wt. % to about 99 wt. %, from about 80 wt. % to about 95 wt. %, from about 80 wt. % to about 85 wt. %, from about 85 wt. % to about 99 wt. %, from about 85 wt. % to about 95 wt. %, from about 85 wt. % to about 90 wt. %, from about 90 wt. % to about 99 wt. %, from about 90 wt. % to about 95 wt. %, or from about 95 wt. % to about 99 wt. %. In some embodiments, the particulate solid may comprise less than 1.0 wt. % of coke when passed to the regeneration unit

As described herein, the majority of the carbon dioxide that is produced in the methods described herein may be produced in the strip zone and subsequently passed out of the strip zone and combined with the one or more olefinic compounds. In particular, in one or more embodiments, at least 80 wt. % of the total coke that is reacted in the strip zone and the regeneration unit may be reacted in the strip zone. For example, according to various embodiments contemplated, at least 85 wt. %, at least 90 wt. %, at least 95 wt. %, or even at least 99 wt. % of the total coke that is reacted in the strip zone and the regeneration unit may be reacted in the strip zone. Additionally, the majority (greater than 50 mol. %) of the carbon dioxide that is produced in the strip zone may be passed out of the strip zone and combined with the one or more olefinic compounds, such as at least 60 mol. %, at least 70 mol. %, 80 mol. %, 90 mol. %, 95 mol. %, or even 99 mol. %. The combination of these two schemes may allow for the vast majority of carbon dioxide produced to be passed out of the reactor system 102 in a stream containing the reaction products, rather than the flue gas stream exiting the regeneration unit 300.

Without being bound by theory, it is believed that by exposing the particulate solid to an oxygen-containing gas in the strip zone 224 the coke that may form on the particulate solid may be primarily combusted within the strip zone 224 with sometimes lesser (such as little to no coke combustion) within the regeneration unit 300. The combustion of coke may produce CO₂ which may require relatively difficult downstream separation and processing steps when mixed with other gases such as hydrocarbons. The product stream may already require downstream processing and separation steps, as such, it may be advantageous to pass much of the CO₂ that is produced through the same stream as the products to utilize the already required downstream processing and separation operations, according to one or more embodiments. Therefore, less CO₂ is passed to the atmosphere via the flue gas.

In one or more embodiments, the addition of an oxygen-containing gas to the strip zone 224 combined with flowing a particulate solid and the oxygen-containing gas in a countercurrent flow pattern through the strip zone 224 may combust a majority of or essentially all of the coke that forms on the particulate solid within the strip zone 224. Such a strip zone 224 configuration may allow the particulate solid to enter the regeneration unit 300 with little or no coke formed on the particulate solid which may reduce or eliminate the production of CO₂ within the regeneration unit 300. In examples where the fuel used in the regeneration unit 300 is hydrogen, the primary component of the exhaust from the regeneration unit 300 may be water which can be safely emitted into the atmosphere limiting the amount of necessary processing of the regeneration unit exhaust and reducing the cost of running the reactor system.

In one or more embodiments, the strip zone 224 may comprise a third gas inlet 530 where a fuel may be injected. In one or more embodiments a fuel may enter the strip zone 224 through the third gas inlet 530. In some embodiments, the third gas inlet 530 may be above the first gas inlet 520 and the second gas inlet 510. In some embodiments, the third gas inlet 530 may be between the first gas inlet 520 and the second gas inlet 510. In some embodiments, the third gas inlet 530 may be beneath the first gas inlet 520 and the second gas inlet 510. In some embodiments, the third gas inlet 530 may be at the same height as the first gas inlet 520 and the second gas inlet 510. In some embodiments, the fuel may comprise hydrogen, methane, ethane, propane, natural gas, or combinations thereof. In some embodiments, the concentration of fuel in the strip zone 224 may be greater than 20 mol. %. In some embodiments, the concentration of fuel in the strip zone 224 may be from about 5 mol. % to about 20 mol. %, from about 5 mol. % to about 15 mol. %, from about 5 mol. % to about 10 mol. %, from about 10 mol. % to about 20 mol. %, from about 10 mol. % to about 15 mol. %, or from about 15 mol. % to about 20 mol. %. In some embodiments, the strip zone 224 may not comprise a third gas inlet 530 (not pictured in FIG. 1).

Now referring to the reactor system 102 of FIG. 2, the reactor system 104 is identical to the reactor system 102 of FIG. 1 except the strip zone comprises a single gas inlet 540. Accordingly, in one or more embodiments oxygen in an oxygen-containing gas and a strip gas may enter the strip zone 224 through gas inlet 540. For example, these streams may be combined upstream of entrance to the strip zone 224. In some embodiments, a mixture of steam and air may enter the strip zone 224 through the gas inlet 540. In some embodiments, air may enter the strip zone 224 through gas inlet 540. In embodiments where the strip zone comprises a single gas inlet 540, the particulate solid and gases within the strip zone 224 may move in a countercurrent flow pattern as the particulate solid moves in a generally downwards direction and the gases move in a generally upwards direction through the strip zone 224.

In additional embodiments, the oxygen and the nitrogen may be passed into the strip zone 224 through a single gas inlet (such as inlet 540) as an air stream. In such embodiments, the air stream encompasses at least nitrogen as the strip gas as well as oxygen, since air includes both nitrogen and oxygen. Such an embodiment may eliminate the need for separate oxygen-containing gas stream and stripping gas streams.

Now referring back to FIG. 1, according to one or more embodiments, the separation device 220 may be a cyclonic separation system, which may include two or more stages of cyclonic separation. In embodiments where the separation device 220 comprises more than one cyclonic separation stages, the first separation device into which the fluidized stream enters is referred to a primary cyclonic separation device. The fluidized effluent from the primary cyclonic separation device may enter into a secondary cyclonic separation device for further separation. Primary cyclonic separation devices may include, for example, primary cyclones, and systems commercially available under the names VSS (commercially available from UOP), LD2 (commercially available from Stone and Webster), and RS2 (commercially available from Stone and Webster). Primary cyclones are described, for example, in U.S. Pat. Nos. 4,579,716; 5,190,650; and 5,275,641, which are each incorporated by reference in their entirety herein. In some separation systems utilizing primary cyclones as the primary cyclonic separation device, one or more set of additional cyclones, e.g. secondary cyclones and tertiary cyclones, are employed for further separation of the particulate solid from the product gas. It should be understood that any primary cyclonic separation device may be used in embodiments of the invention.

Still referring to FIG. 1, the separated particulate solid is passed from the particulate solid separation section 210 to the combustor 350. In the combustor 350, the particulate solid may be processed by, for example, combustion with oxygen. For example, and without limitation, the particulate solid may be de-coked and/or fuel may be combusted to heat the particulate solid. The particulate solid is then passed out of the combustor 350 and through the riser 330 to a riser termination separator 378, where the gas and solid components from the riser 330 are at least partially separated. The vapor and remaining solids are transported to a secondary separation device 320 in the particulate solid separation section 310 where the remaining particulate solid is separated from the gases from the particulate solid processing (e.g., gases emitted by combustion of spent particulate solid or fuel, referred to herein as flue gas). The flue gas may pass out of the regeneration unit 300 via outlet pipe 432. In one or more embodiments the flue gas that passes out of the regeneration unit 300 via outlet pipe 432 may comprise less than 5 mol. % carbon dioxide (for example, in additional embodiments, less than or equal to 4 mol. %, 3 mol. %, 2 mol. %, 1 mol. %, or even 0.5 mol. % or 0.25 mol. %). In some embodiments, the flue gas that passes out of the regeneration unit 300 via outlet pipe 432 may not comprise carbon dioxide. The separated particulate solid is then passed through the oxygen treatment zone 370 within the particulate solid separation section 310 to the upstream reactor section 250 via standpipe 424 and transport riser 430, where it is further utilized in a catalytic reaction. Thus, the particulate solid, in operation, may cycle between the reactor portion 200 and the regeneration unit 300. In general, the processed chemical streams, including the feed streams and product streams may be gaseous, and the particulate solid may be fluidized particulate solid.

Referring now to the regeneration unit 300, as depicted in FIG. 1, the combustor 350 of the regeneration unit 300 may include one or more lower reactor portion inlet ports 352 and may be in fluid communication with the riser 330. Oxygen-containing gas, such as air, may be passed through pipe 428 into the combustor 350. The combustor 350 may be in fluid communication with the particulate solid separation section 210 via standpipe 426, which may supply spent particulate solid from the reactor portion 200 to the regeneration unit 300 for regeneration. The combustor 350 and riser 330, collectively referred to as the particulate solid combustion reactor 302, may operate with similar or identical fluidization regimes as to what was disclosed with respect to the upstream reactor section 250 and downstream reactor section 230 of the reactor portion 200. That is, the combustor 350 may operate as a fluidized bed, such as in a fast fluidized, turbulent, or bubbling bed upflow reactor, while the riser 330 may operate in more of a plug flow manner, such as in a riser reactor. Geometries as described with respect to the upstream reactor section 250 and downstream reactor section 230 may equally apply to the combustor 350 and riser 330. Additionally, the combustor 350 may also include a fuel inlet 354, which may supply a fuel, such as a hydrocarbon stream or hydrogen, to the combustor 350.

As described in one or more embodiments, following separation of flue gas from particulate solid in the riser termination separator 378 and secondary separation device 320, treatment of the processed particulate solid with an oxygen-containing gas is conducted in the oxygen treatment zone 370. In some embodiments, the oxygen treatment zone 370 includes a fluid solids contacting device. The fluid solids contacting device may include baffles or grid structures to facilitate contact of the processed particulate solid with the oxygen-containing gas. Examples of fluid solid contacting devices are described in further detail in U.S. Pat. Nos. 9,827,543 and 9,815,040. The fluidization regime within the oxygen treatment zone 370 may be bubbling bed type fluidization. The oxygen treatment zone 370 may include an oxygen-containing gas inlet 372, which may supply an oxygen-containing gas to the oxygen treatment zone 370 for oxygen treatment of the particulate solid.

In non-limiting examples, the reactor system 102 described herein may be utilized to produce olefinic compounds from hydrocarbon feed streams. As used herein, the term “olefinic compounds” refers to hydrocarbons having one or more carbon-carbon double bonds apart from the formal double bonds in aromatic compounds. For example, ethylene and styrene are olefinic compounds, but ethylbenzene would not be an olefinic compound as the only double bonds present in ethylbenzene are formal double bonds present as part of the aromatic structure. Olefinic compounds may be produced from a variety of hydrocarbon feed streams by utilizing different reaction mechanisms. For example, olefinic compounds may be produced by at least dehydrogenation reactions, cracking reactions, dehydration reactions, and methanol-to-olefin reactions. These reaction types may utilize different feed streams and different particulate solids to produce olefinic compounds. It should be understood that when “catalysts” are referred to herein, they may equally refer to the particulate solid referenced with respect to the system of FIG. 1 and FIG. 2.

According to one or more embodiments, the reaction may be a dehydrogenation reaction. According to such embodiments, the one or more hydrocarbons may be a hydrocarbon feed stream the hydrocarbon feed stream may comprise one or more of ethylbenzene, ethane, propane, n-butane, and i-butane. In one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethylbenzene. In one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of propane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of n-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of the sum of ethylbenzene, ethane, propane, n-butane, and i-butane.

In one or more embodiments, the dehydrogenation reaction may utilize gallium and/or platinum particulate solids as a catalyst. In such embodiments, the particulate solids may comprise a gallium and/or platinum catalyst. As described herein, a gallium and/or platinum catalyst comprises gallium, platinum, or both. The gallium and/or platinum catalyst may be carried by an alumina or alumina silica support, and may optionally comprise potassium. Such gallium and/or platinum catalysts are disclosed in U.S. Pat. No. 8,669,406, which is incorporated herein by reference in its entirety. However, it should be understood that other suitable catalysts may be utilized to perform the dehydrogenation reaction.

In one or more embodiments, the reaction mechanism may be dehydrogenation followed by combustion (in the same chamber). In such embodiments, a dehydrogenation reaction may produce hydrogen as a byproduct, and an oxygen carrier material may contact the hydrogen and promote combustion of the hydrogen, forming water. Examples of such reaction mechanisms, which are contemplated as possible reactions mechanisms for the systems and methods described herein, are disclosed in WO 2020/046978 and U.S. Pat. Pub. No. 2021/0292259 the teachings of which are incorporated by reference in their entireties herein.

In one or more embodiments, the particulate solid may comprise an oxygen-carrier material and a dehydrogenation catalyst material. In some embodiments, the particulate solid may consist essentially of the oxygen-carrier material. As described herein, “consists essentially of” refers to materials with less than 1 wt. % of the non-recited materials (i.e., consisting essentially of A means A is at least 99 wt. % of the composition). In some embodiments, the particulate solid may not comprise a dehydrogenation catalyst material. In some embodiments, the oxygen-carrier material and the dehydrogenation catalyst material may be separate particles of the particulate solid. In some embodiments, the oxygen-carrier material and the dehydrogenation catalyst may be contained in the same particles of the particulate solid.

In embodiments where the particulate solid comprises a dehydrogenation catalyst, the dehydrogenation of the one or more hydrocarbons may be at least partially by catalytic dehydrogenation. Catalytic dehydrogenation is the dehydrogenation of a hydrocarbon that is promoted by the use of a dehydrogenation catalyst. In embodiments, where the particulate solid does not comprise a dehydrogenation catalyst the dehydrogenation reaction may be a non-catalytic thermal dehydrogenation reaction. Non-catalytic thermal dehydrogenation refers to the dehydrogenation of a hydrocarbon that occurs without the use of a dehydrogenation catalyst and instead may occur because of high temperature, pressure or combinations thereof.

In some embodiments, the particulate solid may comprise a “dual-purpose material” that may act as both a dehydrogenation catalyst as well as an oxygen-carrier material. It should be understood that, in at least the embodiments described herein where an oxygen-carrier material and a dehydrogenation catalyst are utilized in the same reaction vessel (such as those of FIG. 1), such a dual-purpose material may be utilized either in replacement or in combination with the oxygen-carrier material of the particulate solid or the dehydrogenation catalyst of the particulate solid.

According to one or more embodiments, the reaction may be a cracking reaction. According to such embodiments, the hydrocarbon feed stream may comprise one or more of naphtha, n-butane, or i-butane. According to one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of naphtha. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of n-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of i-butane. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of the sum of naphtha, n-butane, and i-butane.

In one or more embodiments, the cracking reaction may utilize one or more zeolites as a catalyst. In such embodiments, the particulate solids may comprise one or more zeolites. In some embodiments, the one or more zeolites utilized in the cracking reaction may comprise a ZSM-5 zeolite. However, it should be understood that other suitable catalysts may be utilized to perform the cracking reaction. For example, suitable catalysts that are commercially available may include Intercat Super Z Excel or Intercat Super Z Exceed. In additional embodiments, the cracking catalyst may comprise, in addition to a catalytically active material, platinum. For example, the cracking catalyst may include from 0.001 wt. % to 0.05 wt. % of platinum. The platinum may be sprayed on as platinum nitrate and calcined at an elevated temperature, such as around 700 °C. Without being bound by theory, it is believed that the addition of platinum to the catalyst may allow for easier combustion of fuels, such as methane.

According to one or more embodiments, the reaction may be a dehydration reaction. According to such embodiments, the hydrocarbon feed stream may comprise one or more of ethanol, propanol, or butanol. According to one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of ethanol. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of propanol. In additional embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of butanol. In additional embodiments, the hydrocarbon feed stream or may comprise at least 50 wt. %, at least 60 wt. %, at least 70 wt. %, at least 80 wt. %, at least 90 wt. %, at least 95 wt. % or even at least 99 wt. % of the sum of ethanol, propanol, and butanol.

In one or more embodiments, the dehydration reaction may utilize one or more acid catalysts. In such embodiments, the particulate solids may comprise one or more acid catalysts. In some embodiments, the one or more acid catalysts utilized in the dehydration reaction may comprise a zeolite (such as ZSM-5 zeolite), alumina, amorphous aluminosilicate, acid clay, or combinations thereof. For example, commercially available alumina catalysts which may be suitable, according to one or more embodiments, include SynDol (available from Scientific Design Company), V200 (available from UOP), or P200 (available from Sasol). Commercially available zeolite catalysts which may be suitable include CBV 8014, CBV 28014 (each available from Zeolyst). Commercially available amorphous aluminosilicate catalysts which may be suitable include silica-alumina catalyst support, grade 135 (available from Sigma Aldrich). However, it should be understood that other suitable catalysts may be utilized to perform the dehydration reaction.

According to one or more embodiments, the reaction may be a methanol-to-olefin reaction. According to such embodiments, the hydrocarbon feed stream may comprise methanol. According to one or more embodiments, the hydrocarbon feed stream may comprise at least 50 wt. %, at least 60 wt. %, at least 70 wt. %, at least 80 wt. %, at least 90 wt. %, at least 95 wt. % or even at least 99 wt. % of methanol.

In one or more embodiments, the methanol-to-olefin reaction may utilize one or more zeolites as a catalyst. In such embodiments, the particulate solids may comprise one or more zeolites. In some embodiments, the one or more zeolites utilized in the methanol-to-olefin reaction may comprise a one or more of a ZSM-5 zeolite or a SAPO-34 zeolite. However, it should be understood that other suitable catalysts may be utilized to perform the methanol-to-olefin reaction.

In one or more embodiments, the particulate solid may be capable of fluidization. In some embodiments, the particulate solid may exhibit properties known in the industry as “Geldart A” or “Geldart B” properties. Particles may be classified as “Group A” or “Group B” according to D. Geldart, Gas Fluidization Technology, John Wiley & Sons (New York, 1986), 34-37; and D. Geldart, “Types of Gas Fluidization,” Powder Technol. 7 (1973 ) 285-292, which are incorporated herein by reference in their entireties.

Group A is understood by those skilled in the art as representing an aeratable powder, having a bubble-free range of fluidization; a high bed expansion; a slow and linear deaeration rate; bubble properties that may include a predominance of splitting/recoalescing bubbles, with a maximum bubble size and large wake; high levels of solids mixing and gas backmixing, assuming equal U−Umf (U is the velocity of the carrier gas, and Umf is the minimum fluidization velocity, typically though not necessarily measured in meters per second, m/s, i.e., there is excess gas velocity); axisymmetric slug properties; and no spouting, except in very shallow beds. The properties listed tend to improve as the mean particle size decreases, assuming equal cfp; or as the <45 micrometers (μm) proportion is increased; or as pressure, temperature, viscosity, and density of the gas increase. In general, the particles may exhibit a small mean particle size and/or low particle density (<1.4 grams per cubic centimeter, g/cm³), fluidize easily, with smooth fluidization at low gas velocities, and may exhibit controlled bubbling with small bubbles at higher gas velocities.+Group B is understood by those skilled in the art as representing a “sand-like” powder that starts bubbling at Umf; that exhibits moderate bed expansion; a fast deaeration; no limits on bubble size; moderate levels of solids mixing and gas backmixing, assuming equal U−Umf; both axisymmetric and asymmetric slugs; and spouting in only shallow beds. These properties tend to improve as mean particle size decreases, but particle size distribution and, with some uncertainty, pressure, temperature, viscosity, or density of gas seem to do little to improve them. In general, most of the particles having a particle size (cfp) of 40 μm<cfp <500 μm when the density (pp) is 1.4<pp<4 g/cm³, and preferably 60 μm<cfp <500 μm when the density (pp) is 4 g/cm³ and 250 μm<cfp<100 μm when the density (pp) is 1g/cm³.

In one or more embodiments, the olefinic compounds may be present in a “product stream” sometimes called an “olefin-containing effluent”. Such a stream exits the reactor system of FIG. 1 and may be subsequently processed. In one or more embodiments, the olefinic compounds may comprise one or more of ethylene, propylene, butylene, or styrene. The term butylene includes any isomers of butylene, such as a-butylene, cis-β-butylene, trans-β-butylene, and isobutylene. In some embodiments, the olefin-containing effluent may comprise at least 50 wt. %, at least 60 wt. %, at least 70 wt. %, at least 80 wt. %, at least 90 wt. %, at least 95 wt. % or even at least 99 wt. % of ethylene. In additional embodiments, the olefin-containing effluent may comprise at least 50 wt. %, at least 60 wt. %, at least 70 wt. %, at least 80 wt. %, at least 90 wt. %, at least 95 wt. % or even at least 99 wt. % of propylene. In additional embodiments, the olefin-containing effluent may comprise at least 50 wt. %, at least 60 wt. %, at least 70 wt. %, at least 80 wt. %, at least 90 wt. %, at least 95 wt. % or even at least 99 wt. % of butylene. In additional embodiments, the olefin-containing effluent may comprise at least 50 wt. %, at least 60 wt. %, at least 70 wt. %, at least 80 wt. %, at least 90 wt. %, at least 95 wt. % or even at least 99 wt. % of styrene. In additional embodiments, the olefin-containing effluent may comprise at least 50 wt. %, at least 60 wt. %, at least 70 wt. %, at least 80 wt. %, at least 90 wt. %, at least 95 wt. % or even at least 99 wt. % of the sum of one or more of ethylene, propylene, butylene, and styrene. The olefin-containing effluent may further comprise unreacted components of the feed stream, as well as other reaction products that are not considered light olefins. The olefinic compounds may be separated from unreacted components in subsequent separation steps.

Several aspect are presently disclosed herein. A first aspect is a method for forming olefinic compounds, the method comprising: contacting a feed stream comprising one or more hydrocarbons with a particulate solid in a reactor, wherein in the reactor: the one or more hydrocarbons are reacted to form one or more olefinic compounds; and coke is formed on the particulate solid; passing the particulate solid from the reactor to a strip zone, wherein: the particulate solid is contacted with a strip gas and oxygen in the strip zone; the strip gas comprises nitrogen, steam, or combinations thereof; in the strip zone, a majority of the coke is reacted with oxygen to form carbon dioxide; the majority of the carbon dioxide produced in the strip zone is passed out of the strip zone and combined with the one or more olefinic compounds; passing the particulate solid from the strip zone to a regeneration unit, wherein the particulate solid is heated by combustion of a fuel in the regeneration unit; and passing at least a portion of the particulate solid from the regeneration unit to the reactor.

Another aspect includes any previous aspect or combination of aspects, wherein at least 80 wt. % of the total coke that is reacted in the strip zone and the regeneration unit is reacted in the strip zone.

Another aspect includes any previous aspect or combination of aspects, wherein the particulate solid comprises less than 1.0 wt. % of coke when passed to the regeneration unit.

Another aspect includes any previous aspect or combination of aspects, wherein the particulate solid moves in a generally downward direction through the strip zone and gases move in a generally upwards direction through the strip zone, such that the particulate solid and gases move in a countercurrent flow pattern through the strip zone.

Another aspect includes any previous aspect or combination of aspects, wherein the oxygen is passed into the strip zone in an oxygen-containing gas, and the oxygen-containing gas and the strip gas enter into the strip zone through separate gas inlets.

Another aspect includes any previous aspect or combination of aspects, wherein the strip zone comprises a first gas inlet, a second gas inlet, and a third gas inlet, and wherein: the oxygen-containing gas enters into the strip zone through the first gas inlet; the strip gas enters into the strip zone through the second gas inlet; a fuel enters into the strip zone through the third gas inlet; the first gas inlet is above the second gas inlet; and the third gas inlet is above the first gas inlet.

Another aspect includes any previous aspect or combination of aspects, wherein the strip gas comprises nitrogen, and the oxygen and the nitrogen are passed into the strip zone through a single gas inlet as an air stream.

Another aspect includes any previous aspect or combination of aspects, wherein the fuel comprises hydrogen.

Another aspect includes any previous aspect or combination of aspects, wherein the amount of carbon dioxide in the flue gas from the regeneration unit is less than or equal to 0.5 mol. % of the flue gas.

Another aspect includes any previous aspect or combination of aspects, wherein a portion of the particulate solid is withdrawn from the strip zone and passed to the reactor without first passing through the regeneration unit.

Another aspect includes any previous aspect or combination of aspects, wherein the reaction in the reactor is a dehydrogenation reaction; the one or more hydrocarbons comprise ethylbenzene, ethane, propane, n-butane, i-butane, or combinations thereof; and the particulate solid comprises a dehydrogenation catalyst, an oxygen-carrier material, or both.

Another aspect includes any previous aspect or combination of aspects, wherein the reaction in the reactor is a non-catalytic thermal dehydrogenation reaction; the one or more hydrocarbons comprise ethylbenzene, ethane, propane, n-butane, i-butane, or combinations thereof; and the particulate solid consists essentially of an oxygen-carrier material.

Another aspect includes any previous aspect or combination of aspects, wherein the reaction in the reactor is a cracking reaction; the one or more hydrocarbons comprise naphtha, n-butane, i-butane, or combinations thereof; and the particulate solid comprises one or more zeolites.

Another aspect includes any previous aspect or combination of aspects, wherein: the reaction in the reactor is a dehydration reaction; the one or more hydrocarbons comprise ethanol, propanol, butanol, or combinations thereof; and the particulate solid comprises one or more acid catalysts.

Another aspect includes any previous aspect or combination of aspects, wherein: the reaction in the reactor is a methanol-to-olefin reaction; the one or more hydrocarbons may comprise methanol; and the particulate solid comprises one or more zeolites.

It will be apparent to those skilled in the art that various modifications and variations can be made to the presently disclosed technology without departing from the spirit and scope of the technology. Since modifications combinations, sub-combinations and variations of the disclosed embodiments incorporating the spirit and substance of the presently disclosed technology may occur to persons skilled in the art, the technology should be construed to include everything within the scope of the appended claims and their equivalents. Additionally, although some aspects of the present disclosure may be identified herein as preferred or particularly advantageous, it is contemplated that the present disclosure is not limited to these aspects.

It is noted that the various details described in this disclosure should not be taken to imply that these details relate to elements that are essential components of the various embodiments described in this disclosure, even in cases where a particular element is illustrated in each of the drawings that accompany the present description. Unless specifically identified as such, no feature disclosed and described herein should be construed as “essential”. Contemplated embodiments of the present technology include those that include some or all of the features of the appended claims.

For the purposes of describing and defining the present disclosure it is noted that the term “about” are utilized in this disclosure to represent the inherent degree of uncertainty that may be attributed to any quantitative comparison, value, measurement, or other representation. The term “about” are also utilized in this disclosure to represent the degree by which a quantitative representation may vary from a stated reference without resulting in a change in the basic function of the subject matter at issue.

In relevant cases, where a composition is described as “comprising” one or more elements, embodiments of that composition “consisting of” or “consisting essentially of” those one or more elements is contemplated herein.

In some embodiments, chemicals or chemical streams are described as “passing” from one system unit or portion of a system unit to another. As described herein, such passing may include direct passing or indirect passing. For example, when passing from “unit A” to “unit B”, direct passing has no intermediate destination between unit A and unit B (i.e., directly through a pipe or other transport passage), and indirect passing may include one or more intermediate destinations between unit A and unit B. For example, a stream passing from unit A to unit B may passed through, without limitation, a heat exchanger, treatment device, etc.

It should be appreciated that compositional ranges of a chemical constituent in a stream or in a reactor should be appreciated as containing, in some embodiments, a mixture of isomers of that constituent. For example, a compositional range specifying butene may include a mixture of various isomers of butene. It should be appreciated that the examples supply compositional ranges for various streams, and that the total amount of isomers of a particular chemical composition can constitute a range.

It is noted that one or more of the following claims and the detailed description utilize the terms “where” or “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that any two quantitative values assigned to a property may constitute a range of that property, and all combinations of ranges formed from all stated quantitative values of a given property are contemplated in this disclosure. Where multiple ranges for a quantitative value are provided, these ranges may be combined to form a broader range, which is contemplated in the embodiments described herein.

1. A method for forming olefinic compounds, the method comprising:
contacting a feed stream comprising one or more hydrocarbons with a particulate solid in a reactor, wherein in the reactor:
the one or more hydrocarbons are reacted to form one or more olefinic compounds; and
coke is formed on the particulate solid;
passing the particulate solid from the reactor to a strip zone, wherein:
the particulate solid is contacted with a strip gas and oxygen in the strip zone;
the strip gas comprises nitrogen, steam, or combinations thereof;
in the strip zone, a majority of the coke is reacted with oxygen to form carbon dioxide;
the majority of the carbon dioxide produced in the strip zone is passed out of the strip zone and combined with the one or more olefinic compounds;
passing the particulate solid from the strip zone to a regeneration unit, wherein the particulate solid is heated by combustion of a fuel in the regeneration unit; and
passing at least a portion of the particulate solid from the regeneration unit to the reactor.

2. The method of claim 1, wherein at least 80 wt. % of the total coke that is reacted in the strip zone and the regeneration unit is reacted in the strip zone.

3. The method of claim 1, wherein the particulate solid comprises less than 1.0 wt. % of coke when passed to the regeneration unit.

4. The method of claim 1, wherein the particulate solid moves in a generally downward direction through the strip zone and gases move in a generally upwards direction through the strip zone, such that the particulate solid and gases move in a countercurrent flow pattern through the strip zone.

5. The method of claim 1, wherein the oxygen is passed into the strip zone in an oxygen-containing gas, and the oxygen-containing gas and the strip gas enter into the strip zone through separate gas inlets.

6. The method of claim 5, wherein the strip zone comprises a first gas inlet, a second gas inlet, and a third gas inlet, and wherein:
the oxygen-containing gas enters into the strip zone through the first gas inlet;
the strip gas enters into the strip zone through the second gas inlet;
a fuel enters into the strip zone through the third gas inlet;
the first gas inlet is above the second gas inlet; and
the third gas inlet is above the first gas inlet.

7. The method of claim 1, wherein the strip gas comprises nitrogen, and the oxygen and the nitrogen are passed into the strip zone through a single gas inlet as an air stream.

8. The method of claim 1, wherein the fuel comprises hydrogen.

9. The method of claim 1, wherein the amount of carbon dioxide in the flue gas from the regeneration unit is less than or equal to 0.5 mol. % of the flue gas.

10. The method of claim 1, wherein a portion of the particulate solid is withdrawn from the strip zone and passed to the reactor without first passing through the regeneration unit.

11. The method of claim 1, wherein:
the reaction in the reactor is a dehydrogenation reaction;
the one or more hydrocarbons comprise ethylbenzene, ethane, propane, n-butane, i-butane, or combinations thereof; and
the particulate solid comprises a dehydrogenation catalyst, an oxygen-carrier material, or both.

12. The method of claim 1, wherein:
the reaction in the reactor is a non-catalytic thermal dehydrogenation reaction;
the one or more hydrocarbons comprise ethylbenzene, ethane, propane, n-butane, i-butane, or combinations thereof; and
the particulate solid consists essentially of an oxygen-carrier material.

13. The method of claim 1, wherein:
the reaction in the reactor is a cracking reaction;
the one or more hydrocarbons comprise naphtha, n-butane, i-butane, or combinations thereof; and
the particulate solid comprises one or more zeolites.

14. The method of claim 1, wherein:
the reaction in the reactor is a dehydration reaction;
the one or more hydrocarbons comprise ethanol, propanol, butanol, or combinations thereof; and
the particulate solid comprises one or more acid catalysts.

15. The method of claim 1, wherein:
the reaction in the reactor is a methanol-to-olefin reaction;
the one or more hydrocarbons may comprise methanol; and
the particulate solid comprises one or more zeolites.
